(19) United States
(12) Patent Application Publication
(10) Pub. No.: US 2007/0031504 A1
(43) Pub. Date: Feb. 8, 2007

(54) Microdevices comprising nanocapsules for controlled delivery of drugs and method of manufacturing same

(75) Inventors: Mao-Jung Maurice Lien, Vancouver (CA); Doug Smith, Vancouver (CA); Arc Rajtar, Coquitlam (CA); Dean-Mo Liu, Richmond (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: MIV Therapeutics Inc., Vancouver (CA)

(21) Appl. No.: 11/207,733

(22) Filed: Aug. 22, 2005

(30) Foreign Application Priority Data
Aug. 2, 2005 (WO) PCT/CA05/01201

(51) Int. Cl. A61K 9/50; A61K 9/16

(52) U.S. Cl. 424/490; 977/906

(57) ABSTRACT

This application relates to a microdevice for delivering drugs to a target location. The microdevice comprises a plurality of nanocapsules assembled together, each having an outer hydrophobic shell and an inner liquid core contained within the shell. At least one drug is dissolved within the inner liquid core. The liquid core comprises a mixture of solvents including at least one solvent for maintaining the hydrophilicity of the inner core (and hence the phase difference between the polymeric shell and the liquid core) and at least one second solvent for enhancing the solubility and bioavailability of the drug. For example, the second solvent may be selected to enable a hydrophobic drug to dissolve within the hydrophilic inner core environment. The inner core may also include a small amount of water-soluble polymer. The application also relates to a method of making the microdevices by formulating a homogenous emulsified solution containing the drug and forming the nanocapsules from the emulsified solution, such as by an atomization process.

### RELATED APPLICATIONS

This application claims priority from Patent Cooperation Treaty Application Serial No. PCT/CA2005/001201 filed 2 Aug. 2005.

### TECHNICAL FIELD

This application relates to nanocapsules formulated for drug delivery purposes.

### BACKGROUND

Many prior art patents and scientific publications describe the synthesis and use of nanocapsules for drug delivery purposes. Depending upon their size, structure and use, nanocapsules are sometimes referred to as microcapsules, micro/nanospheres, micro/nano particles, micromicelles and other similar terms. As reviewed by J. H. Park et al. in “Biodegradable Polymers for Microencapsulation of Drugs”, Molecules 2005 10, 146-161, various techniques are known for encapsulating drugs for controlled delivery. The factors responsible for regulating the drug release rate include the physicochemical properties of the drugs, degradation rate of polymers, and the morphology and size of the microparticles.

Most encapsulation processes utilizing biodegradable polymers are designed to produce single particles rather than groups or assemblies of particles or capsules. Patent Cooperation Treaty publication WO0296368 dated 5 Dec. 2002 describes the encapsulation of nanosuspensions into multivesicular liposomes rather than polymer shells. Q. Ye et al. in “DepoFoam™ technology: a vehicle for controlled delivery of protein and peptide drugs”, Journal of Controlled Release, 64, 155-166, 2000 describe similar technology utilizing liposomes rather synthetic polymers.

In prior art nanocapsules, the encapsulated drug is often in a solid phase rather than a liquid phase. In cases where a liquid core is provided, the encapsulated drug is typically hydrophilic and is produced by a water in oil emulsification process. For example, Japanese patent publication JP2003171264 dated 17 Jun. 2003 provides a method for obtaining sustained release microcapsules by means of an emulsification process. The method employs a water-in-oil emulsion that is produced by using a solution containing a water-soluble drug as an inner aqueous phase and a solution containing a polymer as an oil phase. The emulsion phase is dispersed in the water phase to produce a water-in-oil type emulsion and the product is dried to obtain the sustained release microcapsules

While such prior art processes are useful, they are often not effective for achieving controlled release of hydrophobic drugs, such as many anti-cancer therapies. The need has therefore arisen for improved techniques for formulating and assembling nanocapsules capable of enhanced the controlled release and bioavailability of both hydrophilic and hydrophobic drugs.

### SUMMARY OF THE INVENTION

In accordance with the invention, a drug delivery microdevice is provided comprising a plurality of nanocapsules assembled together. In one embodiment of the invention each of the nanocapsules comprise a hydrophobic outer polymeric shell and a hydrophilic inner liquid core located within the polymeric shell and containing at least one drug dissolved therein. The liquid core includes a mixture of at least one first solvent to maintain the hydrophilicity of the inner core and at least one second solvent to enhance the solubility of the drug in the liquid core.

A method of manufacturing a drug delivery device comprising a plurality of nanocapsules is also disclosed, the method comprising:
(a) providing a first solution comprising at least one drug dissolved in one or more first solvents;
(b) providing a second solution comprising a first polymer dissolved in one or more second solvents;
(c) combining the first solution and the second solution to form an emulsified solution comprising a plurality of closed-cell nanocapsules each having an outer polymeric shell and an inner liquid core containing the at least one drug; and
(d) assembling the nanocapsules to form the drug delivery device.

The application also describes the use of the drug delivery microdevice to deliver drugs to a target location, such as an administration site in vivo.

### BRIEF DESCRIPTION OF DRAWINGS

In drawings which illustrate embodiments of the invention, but which should not be construed as restricting the spirit or scope of the invention in any way,

FIG. 1 is a schematic view of a microdevice comprising a plurality of nanocapsules assembled together in accordance with the invention.

FIGS. 2 is a schematic view showing an atomization process for manufacturing the microdevice of FIG. 1.

FIG. 3 is a scanning electron microscopy (SEM) photograph showing a plurality of discrete microdevices configured in accordance with the invention.

FIG. 4 is a further SEM photograph showing a plurality microdevices.

FIG. 5 is a graph showing a representative drug release profile for a multi-layer microdevice.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

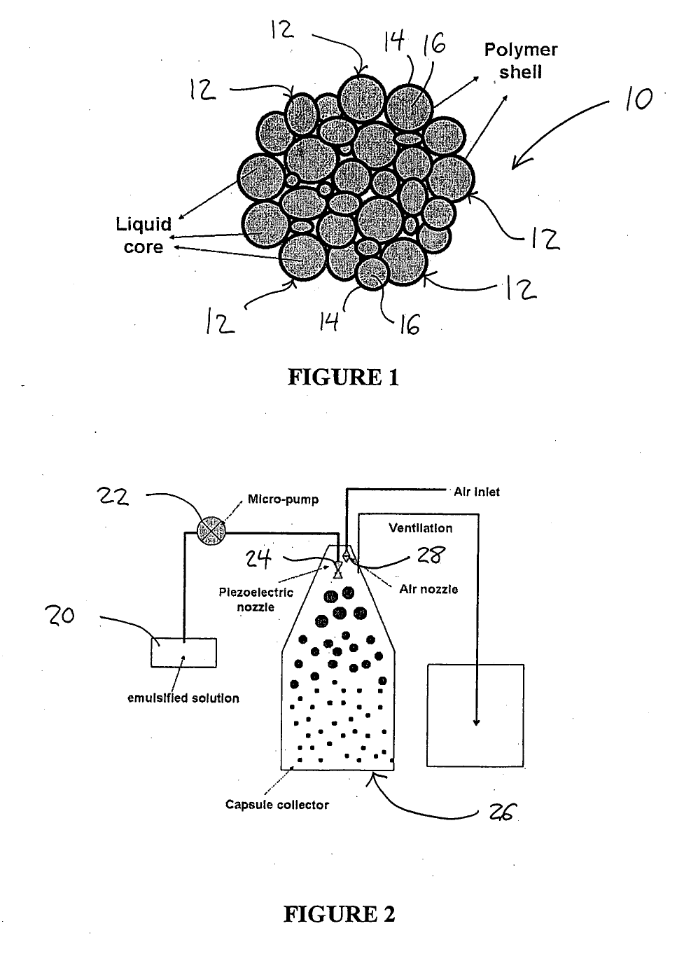

As shown in FIG. 1, this application relates to a drug delivery microdevice 10 comprising a plurality of nanocapsules 12 assembled together. Each nanocapsule 12 includes an outer shell 14 and an inner core 16. As described in detail below, in one embodiment of the invention outer shell 14 is formed from a hydrophobic polymer and inner core 16 comprises at least one drug dissolved in a hydrophilic liquid phase. Each nanocapsule is configured to maintain a distinct interface between the hydrophobic polymeric shell 14 and the hydrophilic liquid core 16, thereby preventing or minimizing interdiffusion therebetween. In use, microdevice 10 can be delivered to a target site in vivo. Outer shell 14 may be configured to biodegrade at the target site to achieve controlled release of drug(s) from inner core 16.

As used in this patent application the term “drug” includes chemical or biological agents intended for therapeutic and/or diagnostic purposes. For example, the term “drug” may include proteins and other biological molecules in addition to conventional pharmaceutical formulations.

As shown in FIG. 1, nanocapsules 12 comprising microdevice 10 are generally spherical in shape (and hence nanocapsules 12 may be referred to as “micropheres” or “nanospheres”). The size and number of nanocapsules 12 may vary without departing from the invention. In some embodiments, each nanocapsule 12 may have a size ranging from about 5 nm to 2,000 nm in diameter. Microdevice 10, comprising a plurality of assembled nanocapsules 12, may have a size ranging from about 20 nm to 5,000 nm in diameter.

Liquid core 16 of each nanocapsule 12 may be configured to deliver either hydrophobic or hydrophilic drugs. To this end, liquid core 16 preferably comprising a mixture of different solvents wherein at least one of the solvents is selected to maintain the hydrophilicity of liquid core 16 and at least another one of the solvents is selected to enhance the solubility of the drug in liquid core 16 and/or to enhance the bioavailability of the drug at the target site. For example, the solvent selected to maintain the hydrophilicity of liquid core 16 (and hence the distinct interface between polymeric shell 14 and liquid core 16) may include ethylene glycol, propylene glycol, butylene glycol, glycerin and water. The solvent selected to enhance the solubility and/or bioavailability of the drug may include lactic acid, glycolic acid, N-dimethylacetamide (DMA), dimethylsulfoxide (DMSO), N,N-diethylnicotinamide (DENA) and diethylformamide (DMF).

By way of example, many therapeutically active drugs are hydrophobic and are not ordinarily soluble or are poorly soluble in a hydrophilic solution. In practice, many such drugs must be administered in high doses in order to be clinically effective. However, this may also increase the risk of deleterious side effects. The present invention enables the effective delivery of water insoluble or poorly soluble drugs by providing a solvent that ensures dissolution of the drug in the liquid phase. For example, drugs such as paclitaxel may be dissolved in liquid core 16 at concentrations between 10-60 weight percent by selecting solvents such as DMSO and DENA. Thus the water solubility of dissolved paclitaxel can be enhanced by 3-4 orders of magnitude as compared with the dried form of crystalline paclitaxel. Other examples of drugs having low water solubility include sirolimus and orathecin. The present invention enhances the bioavailability and therapeutical efficacy of such hydrophobic drugs.

By way of another example, some drugs, such as proteins, are hydrophilic. Such drugs may also be readily dissolved in liquid core 16. As discussed above, apart from the solvent or solvents maintaining the hydrophilicity of liquid core 16, other solvent(s) may be selected to enhance the bioavailability of the drug, including hydrophilic drugs. For example, the solvent(s) may be selected to improve tissue absorption and accordingly enhance therapeutic efficacy.

Liquid core 16 of each nanocapsule 12 may also optionally include a small amount of a water-soluble polymer. The polymer may be present, for example, at a concentration of less than 10 weight percent. In one embodiment, the polymer is present in a concentration of less than 3 weight percent. Suitable polymers include polyvinyl alcohol, poly(acrylic acid), low-molecular poly(ethylene glycol), low molecular poly(propylene glycol), chitosan, gelatin, hyaluronic acid, alginates, cellouse and its derivatives, dextrans and mixtures thereof. The primary purpose of the water-soluble polymer is to act as a surfactant and stabilizer.

Polymeric shell 14 of each nanocapsule 12 is formed from a thin layer of one or more hydrophobic polymers, which may either biodegradable or non-biodegradable. For example, suitable biodegradable polymers include polylactide, polyglycolide, poly(lactide-co-gylcolide), polysulfone, polycaprolactone and combinations thereof. Suitable non-biodegradable polymers include poly(ethylene-vinyl acetate), polyanhydrides, poly(alkylacrylate), polyethylene oxide, and copolymer of polyethylene oxide-poly(propylene oxide), polyurethanes, polysiloxanes and combinations thereof. As described further below, the polymer(s) forming outer shell 14 of each nanocapsule may be derived from a hydrophobic solution in an emulsification process. For example, the polymer(s) may be dissolved in a solution comprising one or more hydrophobic solvents, such as methylene dichloride, methylene trichloride, chloroform, hexanes, and heptanes or mixtures thereof.

One possible process for manufacturing microdevices 10 is shown in FIG. 2. The first step in the process is to form a homogenous emulsified solution 20 containing the drug or drugs of interest. This is accomplished by forming a first solution containing the drug dissolved in the hydrophilic solvents as described above. Optionally a small amount of water-soluble polymer as described above may also be dissolved in the first solution. A second solution comprising a water-insoluble polymer dissolved in one or more hydrophobic solvents is also formed. The first solution (i.e. the dispersed phase) is then dispersed in the second solution (i.e. the continuous phase) by means well-known in the art, such as by vigorous stirring using a homogenizer. The resultant homogenous emulsified solution is stable and will not readily coalesce, even when stored for prolonged periods of time.

As shown schematically in FIG. 2, the homogenous emulsified solution 20 may then be subjected to atomization to form nanocapsules 12 and hence microdevices 10. In particular, the emulsified solution 20 is conveyed by means of a micro-pump 22 to a piezoelectric nozzle 24 mounted within an upper portion of a collector 26. The emulsified solution 20 is instantly atomized into small droplets as it emerges from nozzle 24. An air inlet 28 is located in an upper portion of collector 26 for conveying the small droplets downwardly. Air inlet 28 may include means for regulating the temperature of the inlet air (typically the control temperature is between ambient and 50 degrees Celsius). At the same time, the system includes a ventilator for evaporating or collecting the hydrophobic solvent from the second solution. The rapid and complete removal of the hydrophobic solvent causes the production of nanocapsules 12, each having an outer hydrophobic shell 14 and an inner hydrophilic inner core 16. The nanocapsules assemble together to form microdevices 10 which may be collected as a powder from a bottom portion of collector 26.

Microdevices 10 do not agglomerate when manufactured according to the above-described process. This is especially critical for those applications where a discrete drug-carrying particulate system is clinically desirable.

The size of the nanocapsules 12 produced by the atomization process of FIG. 2 depends upon various factors including the concentration and viscosity of the emulsified solution 20. For example, the lower the concentration and viscosity of the emulsified phase, the smaller the resulting nanocapsules 12 produced. The size distribution of the resulting capsules is thus highly controllable. In different embodiments of the invention the polymer shell 14 of nanocapsules 12 prepared in accordance with the invention may vary between about 5 to 95 weight percent of the total mass of nanocapsules. Liquid cores 16 may accordingly vary between about 95 to 5 weight percent of the total mass of nanocapsules 12.

As will be apparent to a person skilled in the art, many other means for manufacturing microdevices 10 could be employed, including other procedures employing emulsification, homogenization, ultrasonication and/or atomization.

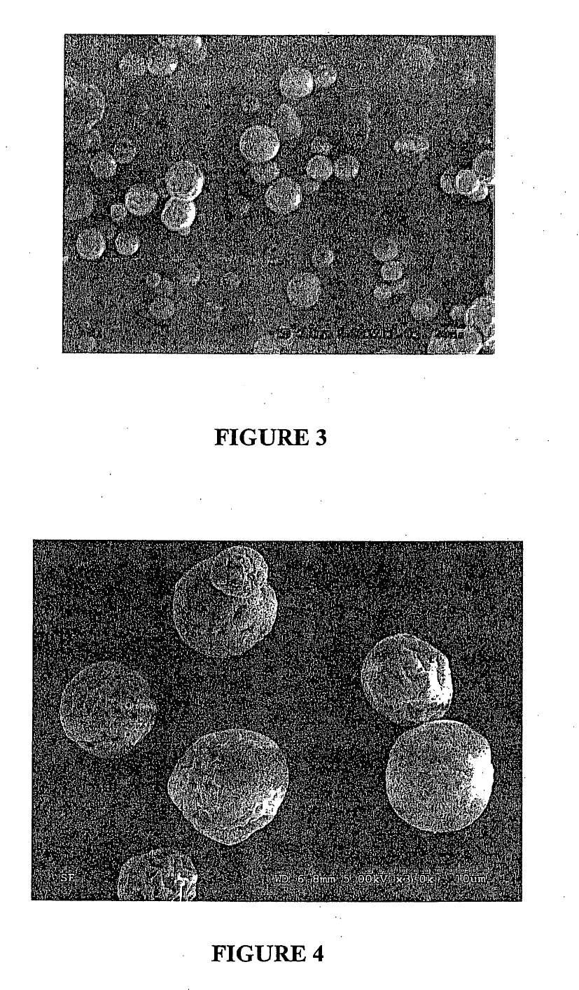

FIGS. 3 and 4 are SEM photographs of microdevices 10 produced in accordance with the invention. The photographs show that each generally spherical microdevice 10 is comprised of an assembly of nanocapsules 12. As best shown in FIG. 4, the high vacuum conditions required for SEM cause bursting of liquid cores 16 of nanocapsules 12 resulting in the formation of small pores visible as artefacts on the SEM photograhps. In the example, of FIG. 4 the pore sizes are between about 80-150 nm in diameter. The size of the pores is indicative of the size of nanocapsules 12 and confirms that such nanocapsules 12 are on the nanometric scale.

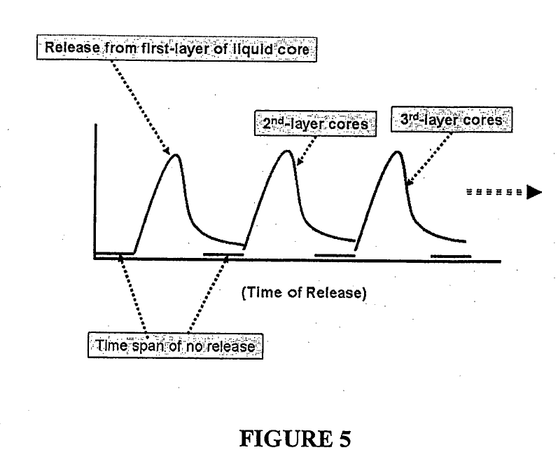

Microdevices 10 constructed in accordance with the invention enable a slow and stepwise drug release profile, as schematically illustrated in FIG. 5. Such a stepwise release is controlled by gradual degradation of the outer shells 14 of successive layers of nanocapsules 12, thereby enabling stepwise release of drug(s) from inner cores 16 into adjacent tissue at the target site. The same degradation-release scenario takes place in a layer-by-layer fashion, from the outermost core layer to inner core layer, until nanocapsules 12 are completely degraded. The time of delayed release can be readily adjusted by selecting the thickness and polymer constituents of outer shells 14. For example the release period may span of several hours, days, weeks or months depending upon the drug(s) and the clinical application.

As will be appreciated by a person skilled in the art, in use microdevices 10 may be administered by various means including injection, inhalation, implantation, ingestion or topical application. The drug(s) may be combined with other pharmaceutically acceptable carriers or adjuvants depending upon the drug(s) and the means of administration. In the case of some applications, microdevices 20 may be applied to another substrate, such as an implantable medical device, for drug delivery purposes. Depending upon the clinical application, each nanocapsule 12 may comprise more than one different drug and/or different nanocapsules 12 may contain different drugs for optimal therapeutic or diagnostic purposes. For example, the outermost nanocapsules 12 may comprise one drug which is initially released in vivo whereas inner nanocapsules may comprise a different drug selected for later release.

### EXAMPLES

The following examples illustrate the invention in further detail although it is appreciated that the invention is not limited to the specific examples.

### Example 1

200 miligrams of paclitaxel is dissolved in a solvent mixture containing 0.8 grams of DMSO, 0.8 grams of ethylene glycol, and 0.2 grams of propylene glycol. The drug-containing solvent mixture is then added dropwisely into a glass vial containing 5 grams of PLGA-methylene chloride solution, wherein the PLGA forms 4 weight percent in the solution. Following vigorous stirring using a homegenizor at a speed of 15,000 rpm for 60 seconds, the emulsified solution is then subjected to microspherization using a commercially available ultrasonic spraying device as illustrated in FIG. 2 to form microdevices 10. Microdevices 10 have a spherical geometry and have a uniform size distribution of 2-5 micrometers. Microdevices 10 can be used as a drug delivery vehicle for biomedical use and, in this example, each microdevice 10 contains 50 weight percent of PLGA shell 14 and 50 weight percent inner core 16, including the drug and hydrophilic solvent mixture.

### Example 2

100 miligrams of paclitaxel is dissolved in a solvent mixture containing 0.2 grams of DMSO, 0.2 grams of DENA, 0.3 grams of ethylene glycol, and 0.2 grams of propylene glycol. The drug-containing solvent mixture is then added dropwisely into a glass vial containing 5 grams of PLGA-methylene chloride solution, wherein the PLGA forms 4 weight percent in the solution. Following vigorous stirring using a homegenizor at a speed of 15,000 rpm for 60 seconds, the emulsified solution is then subjected to microspherization through a commercially available ultrasonic spraying device as illustrated in FIG. 2 to form microdevices 10. In this Example, each microdevice 10 contains 67 weight percent of PLGA shell 14 and 33 weight percent inner core 16, including the drug and hydrophilic solvent mixture. Scanning electron microscopy analysis, as illustrated in FIG. 4, shows the resulting microdevice 10 is an assembly of nanocapsules 12. As indicated above, the size of the pores on the SEM photo of FIG. 4 confirms that nanocapsules 12 are on the nanometric scale, within the range of about 80-150 nm in diameter.

### Example 3

200 miligrams of paclitaxel is dissolved in a solvent mixture containing 0.4 grams of DMSO, 0.4 grams of DENA, 0.8 grams of ethylene glycol, and 0.2 grams of propylene glycol. A small amount of polyelectrolyte poly(acrylic acid) (molecular weight from 2,000 to 450,000) and/or polyethylene glycol (molecular weight of 200, 400, and 2000), corresponding to 2.3 weight percent on the weight of the drug-containing solvent, is dissolved. The drug-containing solvent mixture is then added dropwisely into a glass vial containing 5 grams of PLGA-methylene chloride solution, wherein the PLGA takes 4 weight percent in the solution. Following vigorous stirring using a homegenizor at a speed of 20,000 rpm for 60 seconds, the emulsified solution is then subjected to microspherization using a commercially available ultrasonic spraying device as illustrated in FIG. 2 to form microdevices 10. The emulsified solution used to form microdevices 10 is homogenous and stable and does not coalesce when stored statically for seven days.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A drug delivery microdevice comprising a plurality of nanocapsules assembled together, each of said nanocapsules comprising:
(a) a hydrophobic outer polymeric shell; and
(b) a hydrophilic inner liquid core located within said polymeric shell and containing at least one drug dissolved in said liquid core, wherein said liquid core comprises a mixture of at least one first solvent to maintain the hydrophilicity of said inner core and at least one second solvent to enhance the solubility of said drug in said liquid core.

2. The drug delivery microdevice as defined in claim 1, wherein said liquid core comprises a water-soluble polymer.

3. The drug delivery microdevice as defined in claim 1, wherein said liquid core is polymer-free.

4. The drug delivery microdevice as defined in claim 2, wherein said polymer is a surfactant.

5. The drug delivery microdevice as defined in claim 2, wherein said polymer is selected from the group consisting of polyvinyl alcohol, poly(acrylic acid), low-molecular poly(ethylene glycol), low molecular poly(propylene glycol), chitosan, gelatin, hyaluronic acid, alginates, cellulose and its derivatives and dextrans.

6. The drug delivery microdevice as defined in claim 5, wherein the concentration of said polymer in said liquid core is less than 10% by weight of said liquid core.

7. The drug delivery microdevice as defined in claim 6, wherein the concentration of said polymer in said liquid core is less than 3% by weight of said liquid core.

8. The drug delivery microdevice as defined in claim 1, wherein said first solvent is selected from the group consisting of ethylene glycol, propylene glycol, butylene glycol, glycerin and water.

9. The drug delivery microdevice as defined in claim 1, wherein said second solvent is selected from the group consisting of lactic acid, glycolic acid, N-dimethylacetamide (DMA), dimethylsulfoxide (DMSO), N,N-diethylnicotinamide (DENA) and diethylformamide (DMF).

10. The drug delivery microdevice as defined in claim 1, wherein said at least one drug is hydrophobic.

11. The drug delivery microdevice as defined in claim 1, wherein said at least one drug is hydrophilic.

12. The drug delivery microdevice as defined in claim 1, wherein said microdevice is generally spherical in shape and has a diameter between approximately 20 nm and 5,000 nm in size.

13. The drug delivery microdevice as defined in claim 1, wherein each of said nanocapsules is generally spherical in shape and has a diameter between approximately 5 nm and 2,000 nm in size.

14. The drug delivery microdevice as defined in claim 1, wherein said polymeric shell is biodegradable and biocompatible.

15. The drug delivery microdevice as defined in claim 14, wherein said polymeric shell is formed from a polymer selected from the group consisting of polylactide, polyglycolide, poly(lactide-co-gylcolide), polysulfone and polycaprolactone.

16. The drug delivery microdevice as defined in claim 1, wherein said polymeric shell is non-biodegradable.

17. The drug delivery microdevice as defined in claim 16, wherein polymeric shell is formed from a polymer selected from the group consisting of poly(ethylene-vinyl acetate), polyanhydrides, poly(alkylacrylate), polyethylene oxide, polyurethanes, polysiloxanes and copolymers of polyethylene oxide-poly(propylene oxide).

18. The drug delivery device as defined in claim 1, wherein said polymeric shell comprises between 5-95 weight percent of the total mass of each of said nanocapsules.

19. The drug delivery microdevice as defined in claim 1, wherein said liquid core comprises a pharmaceutically effective carrier for said at least one drug.

20. The drug delivery microdevice as defined in claim 1, wherein said microdevice delivers multiple drugs, wherein different ones of said nanocapsules contain different ones of said multiple drugs.

21. The drug delivery microdevice as defined in claim 1, wherein said at least one drug is insoluble or poorly soluble in water.

22. The drug delivery microdevice as defined in claim 1, wherein said at least one drug is water-soluble.

23. The microdevice as defined in claim 1, wherein said microdevice comprises multiple layers of said nanocapsules.

24. The microdevice as defined in claim 1, further comprising a substrate on to which said nanocapsules are applied.

25. The use of the microdevice as defined in claim 1 for delivery of said at least one drug to a delivery site in a subject comprising:
(a) administering said microdevice to said subject; and
(b) allowing said polymeric shell of at least some of said nanocapsules to degrade, thereby resulting in timed release of said at least one drug from said liquid core at said delivery site.

26. The use as defined in claim 25, wherein said administering is selected from the group consisting of injecting, inhaling, implanting, ingesting and topically applying said microdevice.

27. The use as defined in claim 25, wherein said at least one drug is released gradually in a step-wise manner during the course of a release period.

28. The use as defined in claim 25, wherein said at least one drug is hydrophobic.

29. The use as defined in claim 25, wherein said at least one drug is hydrophilic.

30. A method of manufacturing a drug delivery device comprising a plurality of nanocapsules comprising:
(a) providing a first solution comprising at least one drug dissolved in one or more first solvents;
(b) providing a second solution comprising a first polymer dissolved in one or more second solvents;
(c) combining said first solution and said second solution to form an emulsified solution comprising a plurality of closed-cell nanocapsules each having an outer polymeric shell and an inner liquid core containing said at least one drug; and
(d) assembling said nanocapsules to form said drug delivery device.

31. The method as defined in claim 30, wherein said one or more first solvents comprise a mixture of at least one first solvent to maintain the hydrophilicity of said inner core and at least one other first solvent to enhance the solubility of said drug in said liquid core.

32. The method as defined in claim 31, wherein said at least one first solvent is selected from the group consisting of ethylene glycol, propylene glycol, butylene glycol, glycerin and water and wherein said at least one other first solvent is selected from the group consisting of lactic acid, glycolic acid, N-dimethylacetamide (DMA), dimethylsulfoxide (DMSO), N,N-diethylnicotinamide (DENA) and diethylformamide (DMF).

33. The method as defined in claim 30, wherein said second solution is selected from the group consisting of methylene dichloride, methylene trichloride, chloroform, hexanes, heptanes, octanes, toluene, xylene, 1,1,1-trichloroethane, and 1,1,2-trichloroethane.

34. The method as defined in claim 33, wherein said first polymer is selected from the group consisting of polylactide, polyglycolide, poly(lactide-co-gylcolide), polysulfone and polycaprolactone.

35. The method as defined in claim 30, wherein said first solution comprises a second polymer selected from the group consisting of polyvinyl alcohol, poly(acrylic acid), low-molecular poly(ethylene glycol) and low molecular poly(propylene glycol).

36. A nanocapsule comprising:
(a) a hydrophobic outer polymeric shell; and
(b) a hydrophilic inner liquid core located within said polymeric shell and containing at least one drug dissolved in said liquid core, wherein said liquid core comprises a mixture of at least one first solvent to maintain the hydrophilicity of said inner core and at least one second solvent to enhance the solubility of said drug in said liquid core.

37. A drug delivery microdevice comprising a plurality of nanocapsules assembled together, each of said nanocapsules comprising:
(a) a hydrophobic outer polymeric shell; and
(b) a hydrophilic inner liquid core located within said polymeric shell and containing at least one drug dissolved in said liquid core, wherein said liquid core comprises a mixture of at least one first solvent to maintain the hydrophilicity of said inner core and at least one second solvent to enhance the bioavailability of said drug.
